(19) United States
(12) Patent Application Publication
(10) Pub. No.: US 2018/0338732 A1
(43) Pub. Date: Nov. 29, 2018

(54) SYSTEMS AND METHODS FOR MONITORING HEART AND LUNG ACTIVITY

(71) Applicant: Board of Regents, The University of Texas System, Austin, TX (US)

(72) Inventors: JUNG-CHIH CHIAO, GRAND PRAIRIE, TX (US); WENYUAN SHI, ROWLETT, TX (US); MAGGIE TJIA, PROTLAND, OR (US)

(21) Appl. No.: 15/761,571

(22) PCT Filed: Sep. 20, 2016
(86) PCT No.: PCT/US16/52678

§ 371 Date: Mar. 20, 2018

Related U.S. Application Data
(60) Provisional application No. 62/221,406, filed on Sep. 21, 2015.

(51) Int. Cl. A61B 5/00; A61B 7/04; A61B 7/00; A61B 5/08; H04R 1/46

(52) U.S. Cl. CPC A61B 5/7267; A61B 7/04; A61B 5/681; A61B 2562/0204; A61B 7/003; A61B 5/08; H04R 1/46; A61B 5/0004

(57) ABSTRACT

In one embodiment, a system for monitoring heart activity includes a wearable monitoring device including a sensor adapted to capture arterial pulse wave sounds, and a computing device configured to receive arterial pulse wave sound data from the wearable monitoring device and estimate heart sounds from the data.

### CROSS-REFERENCE TO RELATED APPLICATION

This application claims priority to co-pending U.S. Provisional Application Ser. No. 62/221,406, filed Sep. 21, 2015, which is hereby incorporated by reference herein in its entirety.

### BACKGROUND

The health of the heart and lungs are traditionally assessed by a physician using a stethoscope that is applied to the chest or back. While the sounds the heart and lungs make can be easily heard with a stethoscope, the acoustic parameters of sounds, and therefore the parameters of operation of the heart and lungs, cannot be accurately identified by human hearing. In addition, such parameters cannot be recorded using a conventional stethoscope for purposes of computer analysis.

The usefulness of the sounds acquired with a stethoscope can be greatly enhanced by digital signal processing. Phonocardiography and digital stethoscopes with mathematical decomposition methods have been developed. However, there is no known system or method available in the market that facilitates the continuous capture and analysis of key parameters, such as the occurrence times and frequencies of heart sounds S1 and S2.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a block diagram of an embodiment of a system for monitoring heart and lung activity.

FIG. 2 is a graph that shows stethoscope signal recordings obtained using an experimental monitoring system.

FIG. 3A is a graph that shows segments of heart sounds within 0.8 s.

FIG. 3B is a graph that shows spectral distributions of the heart sounds of FIG. 3A.

FIG. 4A is a graph that shows spectral contour lines.

FIG. 4B is a graph that shows a three-dimensional time-frequency mesh of heart sounds acquired using the disclosed methods.

FIG. 5A is a graph that shows waveforms within 4 s.

FIG. 5B is a graph that shows the spectral contour lines of the waveforms of FIG. 5A.

FIG. 6 is a graph that shows a stethoscope signal recording of lung sounds.

FIG. 7A is a graph that shows segments of lung sound recordings for 2 s.

FIG. 7B is a graph that shows spectral distributions of the segments of FIG. 7A.

FIG. 7C is a graph that shows spectral contour lines of lung sounds acquired using the disclosed methods.

FIG. 8 is a block diagram of an experimental apparatus for comparing heart sounds and arterial pulse wave sounds.

FIG. 9A is a graph of heart sounds obtained from the chest.

FIG. 9B is a graph of arterial pulse wave sounds obtained from the subclavian artery.

FIG. 9C is a graph of arterial pulse wave sounds obtained from the brachial artery.

FIG. 9D is a graph of arterial pulse wave sounds obtained from the radial artery.

FIG. 10 is a block diagram of a travel model of pulse wave sounds in blood vessels.

FIG. 11 is a block diagram of an artificial neural network for training an inverse attenuation function.

FIG. 12 is a graph of arterial pulse wave sounds obtained from the radial artery at the wrist used as the input to a trained network.

FIG. 13 is a graph of heart sounds estimated by the trained network using the arterial pulse wave sounds of FIG. 12.

FIG. 14 is a block diagram of an embodiment of a system for monitoring heart activity.

FIG. 15 is a graph of arterial pulse wave sounds obtained from the radial artery at the wrist with a wearable monitoring device.

FIG. 16 is a graph of heart sounds estimated by the trained network using the arterial pulse wave sounds of FIG. 15.

### DETAILED DESCRIPTION

Disclosed herein are systems and methods for monitoring heart and/or lung activity. In some embodiments, the systems include a monitoring device that can be worn on the chest for continuous monitoring of heart and lung sounds. These sounds can be transmitted to another device, such as a smart phone or a computer, for recordation and analysis for the purpose of diagnosing the condition of the heart and/or lungs. In other embodiments, the systems include a monitoring device that can be worn on the body at a location at which the sounds of the individual's arterial pulse can be continuously monitored, such as the wrist. These sounds can also be transmitted to another device for recordation and analysis. Such analysis can comprise processing the pulse wave sounds to estimate the sounds of the heart.

In the following disclosure, various specific embodiments are described. It is to be understood that those embodiments are example implementations of the disclosed inventions and that alternative embodiments are possible. All such embodiments are intended to fall within the scope of this disclosure.

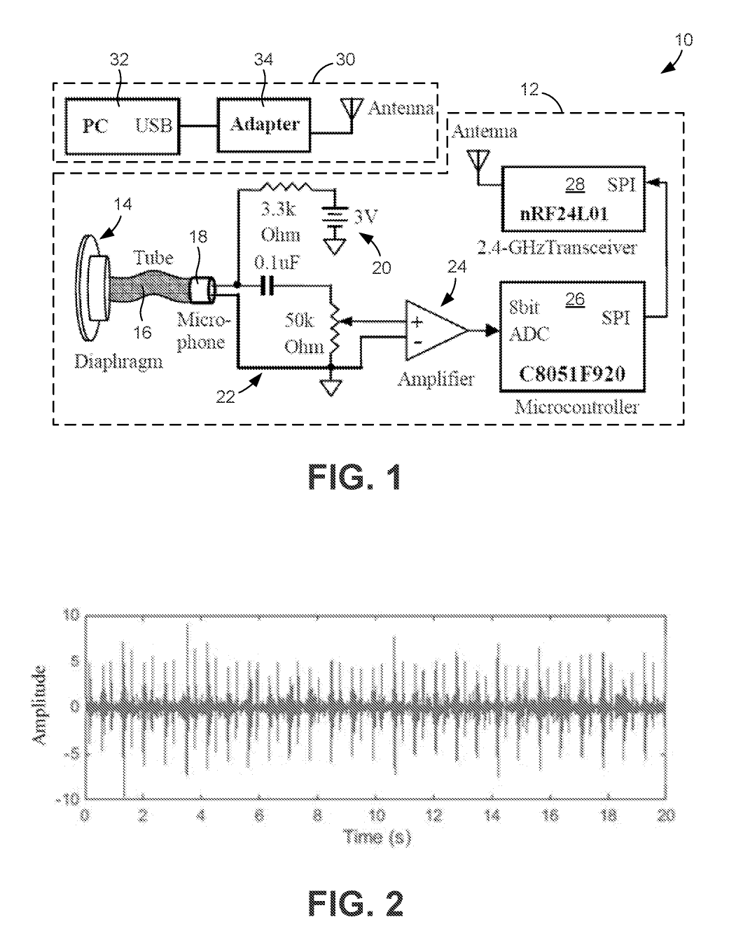

FIG. 1 illustrates an embodiment of a system 10 for monitoring heart and lung activity. As shown in the figure, the system 10 includes a wearable monitoring device 12, which can be worn on the chest for continuous monitoring. The monitoring device 12 can have a small form factor of, for example, approximately 1.5 cm×4 cm, and includes a stethoscope head 14, which can be secured (e.g., taped) to the patient's chest. A tube 16 is connected at a first end to the head 14 and at a second end to a microphone 18. With this configuration, sounds picked up from the chest cavity by the head 14 can travel through the tube 16 to the microphone 18, which is powered by an onboard power source 20, such as a battery. The sounds received by the microphone 18 pass through an electrical circuit 22 to an amplifier 24, which provides an amplified analog signal to a microcontroller 26.

The microcontroller 26 converts the analog signal to a digital signal and provides the digital signal to a radio frequency (RF) transceiver 28 that is adapted to wirelessly transmit the digital signal to a computing device 30. In the example embodiment of FIG. 1, the computing device 30 is a personal computer (PC) 32 that receives the signal using an attached wireless adapter 34. More generally, however, the computing device 30 can comprise any device that is capable of receiving the digital signal and storing it. In some embodiments, the computing device 30 is a portable computing device, such as a laptop computer, tablet, or smart phone, so that it can be carried with the user to enable long-term data collection. In some embodiments, the computing device further comprises software/firmware configured to analyze the digital signal to evaluate the functioning of the patient's heart and/or lungs. Examples of such analysis are described below.

An experimental system similar to that described above in relation to FIG. 1 was constructed for testing purposes. The system comprised a PUM-5250 microphone (PUI Audio, Inc.), operational amplifiers, a 2.4 GHz nRF24L01 (Nordic Semiconductor) wireless transceiver, a C8051F920 microcontroller (Silicon Labs) with 9600 samples/sec and 8-bit resolution ADC, and a USB wireless adapter. The microphone was powered by a 3 V battery and the signals of the heart or lung sounds were connected to the inputs in the wireless module. The analog signals were amplified and converted to 8-bit digital signals by the microcontroller. The digital signals were then transferred to the wireless transceiver by serial peripheral interface (SPI) communication. A 2.4-GHz radio was utilized for broadcast and a wireless adapter received signals into a USB port of a PC.

The stethoscope head was lightly pressed over the aortic region of the chest of a test subject. Heart sounds were recorded by the microphone, which was connected by the tube to the head. The microphone voltage signal was then amplified, wireless transmitted, recorded, and displayed on the PC in real time.

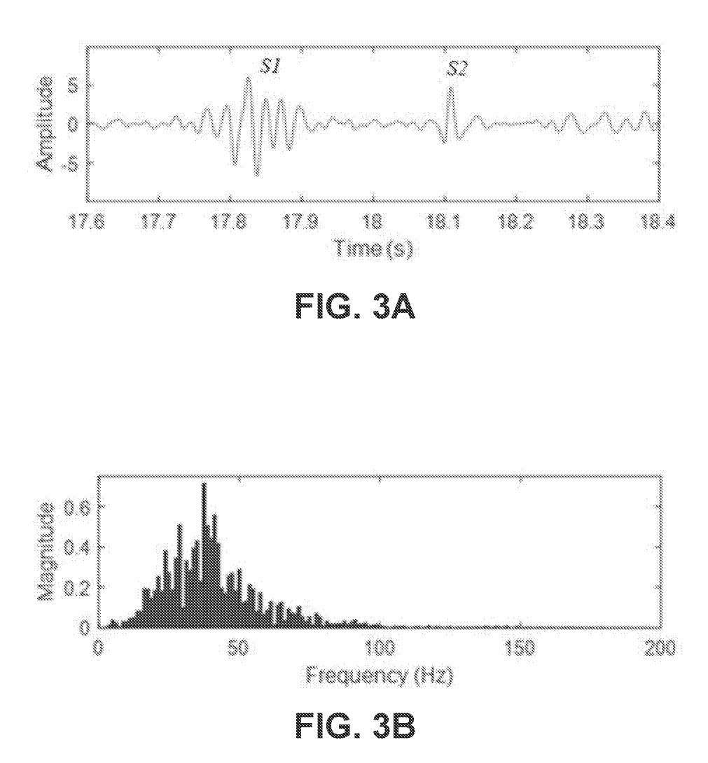

Since the primary heart sounds S1 and S2 occur within a frequency range of 20 to 200 Hz, a Butterworth band-pass filter was used to filter out unwanted signals. The resulting signal shapes are shown in FIG. 2. A normalized segment of the heart sounds is shown in FIG. 3A, which includes two heart sounds, S1 and S2, within a 0.8-s period, while the spectral distributions of the signals are shown in FIG. 3B.

To express the frequency information of heart sounds with time lapse, spectral distributions in the time-frequency domain of the heart sounds were calculated by the discrete short-time Fourier transform (STFT):

$$S ( n , k ) = S ( n , {ω}_{k} ) {\vert}_{{ω}_{k} = \frac{2 π k}{N}} = {\sum }_{m = - ∞}^{+ ∞} s ( m ) W ( n - m ) {e}^{- j \frac{2 π k}{N} m} \qquad ( 1 )$$

where N is number of total samples, frequency ωₖ=2πk/N, 2π/N is the frequency sampling interval, and W is the Hamming window function, which is defined in Equation (2):

$$W ( n ) = 0.54 - 4.46 cos ( \frac{2 π n}{N - 1} ) 0 \leq n \leq N - 1 \qquad ( 2 )$$

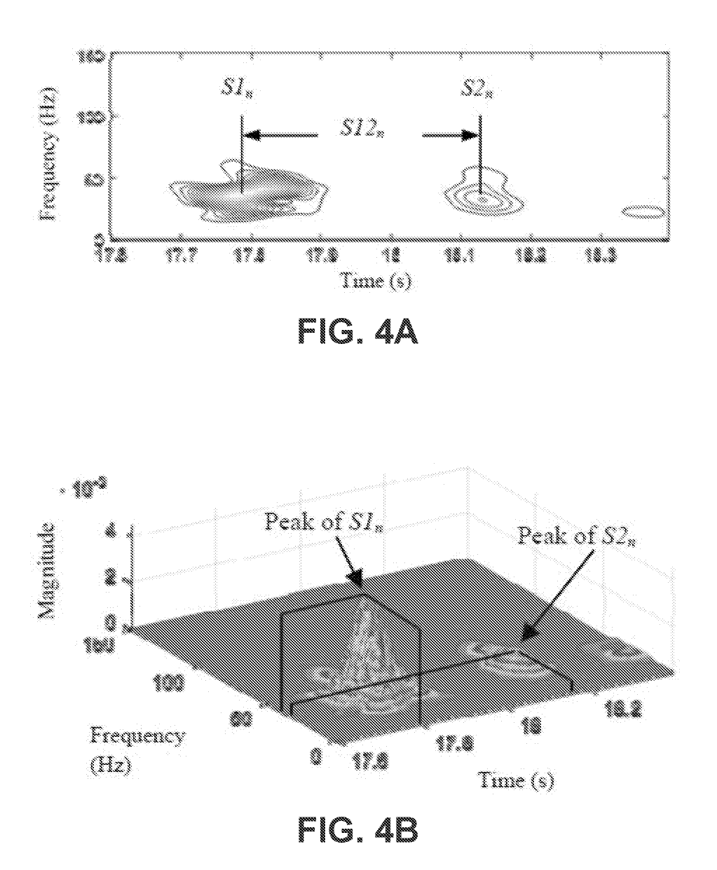

Applying the Hamming window function with a length of N, the heart sound signal s(τ) was divided into segments of length N. The spectral contour lines and three-dimensional time-frequency mesh of the recorded heart sounds within 0.8 s are plotted in FIG. 4.

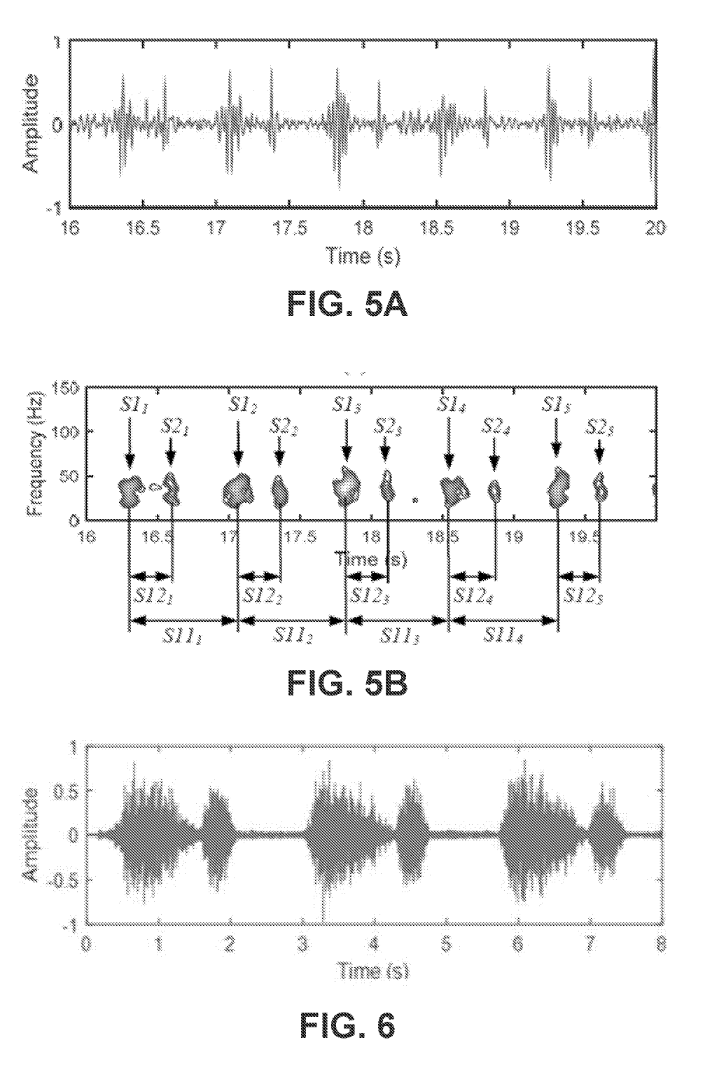

The S1 and S2 acoustic properties can reveal the strength, or weakness, of the myocardial systole and the atrioventricular valve functions. S1 and S2 oscillation frequencies are different for each person. Because the amplitudes of S1 and S2 oscillate in short periods and the frequency components of S1 and S2 are distributed in a wide range, it is rarely reported how to precisely determine heart sound acoustic parameters. A reasonable assumption is that the exact occurrences in time and their frequencies of S1 and S2 are at spectral magnitude peaks in the time-frequency domain. Using the discrete STFT of the heart sounds, occurrence times and frequency components can be obtained by projecting the S1 and S2 peaks onto the time axis and frequency axis as shown in FIG. 4B. The heart sound acoustic parameters can then continuously be derived. In FIG. 5A, four seconds of data were extracted. As shown in FIG. 5B, there are 5 spectral peaks of S1ₙ and S2ₙ in the time-frequency domain, where n=1, 2, 3, 4, 5, occurring within 4 s. Occurrence times and frequencies of the peaks in the spectral contour lines were calculated, as listed in Table 1. Mean values and standard deviations of S1 and S2 sound frequencies were calculated. The time interval between S1ₙ and S1ₙ₊₁ defined as S11ₙ is shown in FIG. 5B.

TABLE 1
Acoustic parameters of S1 and S2

| | S1ₙ | | | | | | |
|---|---|---|---|---|---|---|---|
| | S1₁ | S1₂ | S1₃ | S1₄ | S1₅ | Mean | Std. |
| Time(s) | 16.29 | 17.05 | 17.82 | 18.56 | 19.30 | N/A | N/A |
| Frequency | 36 | 37 | 38 | 35 | 34 | 36.00 | 1.58 |
| (Hz) | | | | | | | |
| | S2ₙ | | | | | | |
| | S2₁ | S2₂ | S2₃ | S2₄ | S2₅ | Mean | Std. |
| Time(s) | 16.59 | 17.5 | 18.11 | 18.86 | 19.61 | N/A | N/A |
| Frequency | 27 | 30 | 34 | 34 | 33 | 31.60 | 3.05 |
| (Hz) | | | | | | | |

TABLE 2
Acoustic parameters of heart sound: time intervals, heart
rates (beats/min), mean values, and standard deviations

| | S1_{n, n+1} | | | | | | |
|---|---|---|---|---|---|---|---|
| | S11₁ | S11₂ | S11₃ | S11₄ | N/A | Mean | Std. |
| Time(s) | 0.754 | 0.771 | 0.737 | 0.747 | N/A | 0.752 | 0.014 |
| Heart rate | 79.57 | 77.83 | 81.40 | 80.29 | N/A | 79.77 | 1.50 |
| | S12ₙ | | | | | | |
| | S12₁ | S12₂ | S12₃ | S12₄ | S12₅ | Mean | Std. |
| Time(s) | 0.298 | 0.304 | 0.291 | 0.308 | 0.308 | 0.302 | 0.007 |

The transient heart rate can be obtained by 60/S11ₙ (beats/min). The time interval between S1ₙ and S2ₙ is defined as S12ₙ. Mean values and standard deviations of transient heart rate and time interval S12ₙ can be calculated, as listed in Table 2. The transient occurrence time of S1 and S2, respective oscillation frequencies, heart rate, and heart sound statistical errors can be continuously extracted in real time using the wireless recording system. These precise acoustic parameters are useful for diagnosis of heart diseases, such as cardiac arrhythmia and heart valve disease. As the wireless stethoscope can be worn for continuous recording, it provides information that can be coordinated with patient's physical activities and emotional behaviors.

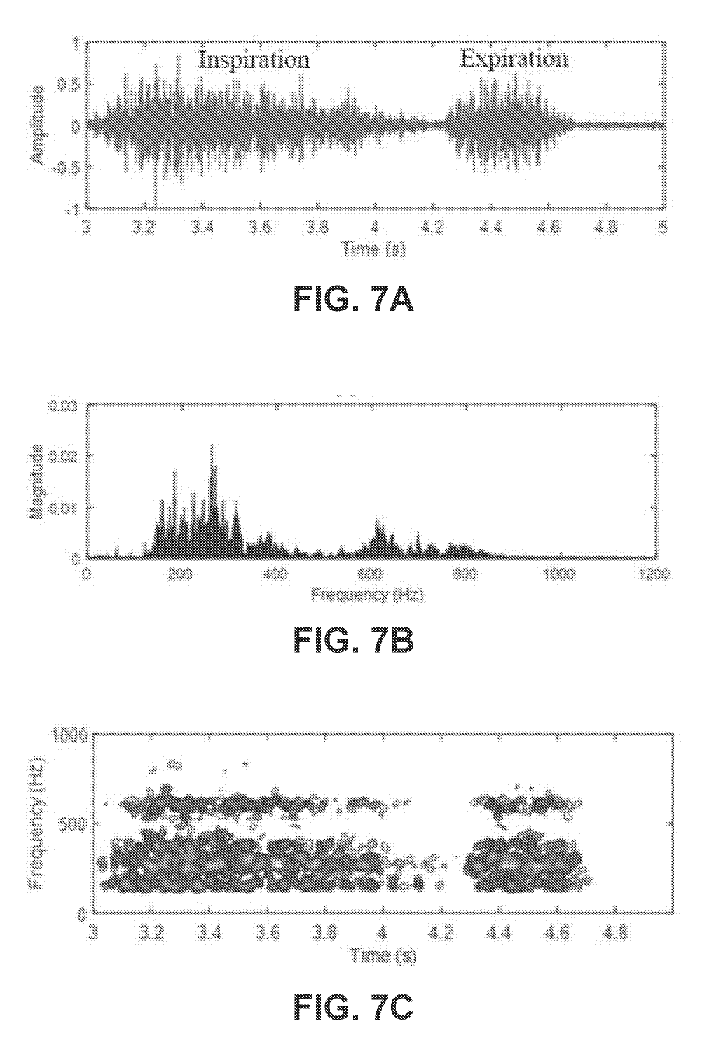

A similar Butterworth band-pass filter with cutoff frequencies at 20 and 1200 Hz was used for lung sound recording. Normalized lung sound signals are shown in FIG. 6. Using the same techniques as those described above, the lung sound recording segment and spectral distributions are shown in FIGS. 7A and 7B. The lung sound data comprised the inspiration and expiration stages. Most of the spectral energy was distributed within 100 to 400 Hz. The spectral distributions in the time-frequency domain calculated using the same discrete STFT are shown in FIG. 7C, providing also the similar quantitative parameters for insights about patient's respiration.

While a system such as that described above can be used to identify important parameters about the functioning of an individual's heart, it can be difficult to use a stethoscope on chest as a wearable sensor due to its size and weight. It would be desirable in at least some cases to have a more convenient wearable device, such a device wearable on the wrist, which can be used to determine the same key parameters that can be detected with the system disclosed above.

When blood flows from the heart to the arteries, the blood pressures and pulse waves change, which is a compound and nonlinear process. Arterial pulse waves can be converted into sound signals just like heart sounds. However, the relationship of the arterial pulse wave sounds to the original heart sounds is complex. If a transfer function of the sound propagation along the artery between two locations were developed to correlate the arterial pulse wave sounds to the heart sounds, parameters such as S1 and S2 could be estimated from the arterial pulse wave sounds without placing a stethoscope on the chest.

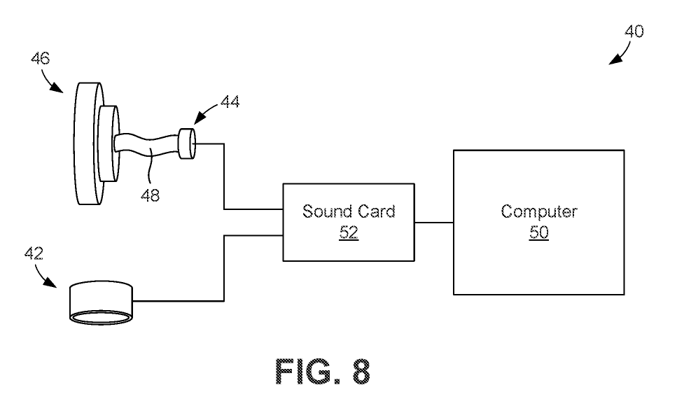

Experiments were performed on a test subject to compare the heart sounds obtained from the chest with arterial pulse wave sounds obtained from the arteries with the aim of identifying a transfer function that can be applied to arterial pulse wave sounds to estimate the heart sounds, such as S1 and S2. FIG. 8 illustrates an apparatus 40 that was used in the experiments. As shown in this figure, the apparatus 40 included a 3D-printed air chamber 42 having a diameter of 15 mm and a thickness of 5 mm that was to be used to capture arterial pulse wave sounds, and a PUM-5250 condenser microphone (PUI Audio, Inc.) 44 connected to a stethoscope head 46 with a tube 48 that was to be used to capture heart sounds. A computer 50 with a sound card 52 was used to simultaneously record both the heart and arterial pulse wave sounds. The sound card gain was set at 25. The analog to digital conversion (ADC) of the sound card was performed at 44,100 samples/second with a 16-bit resolution.

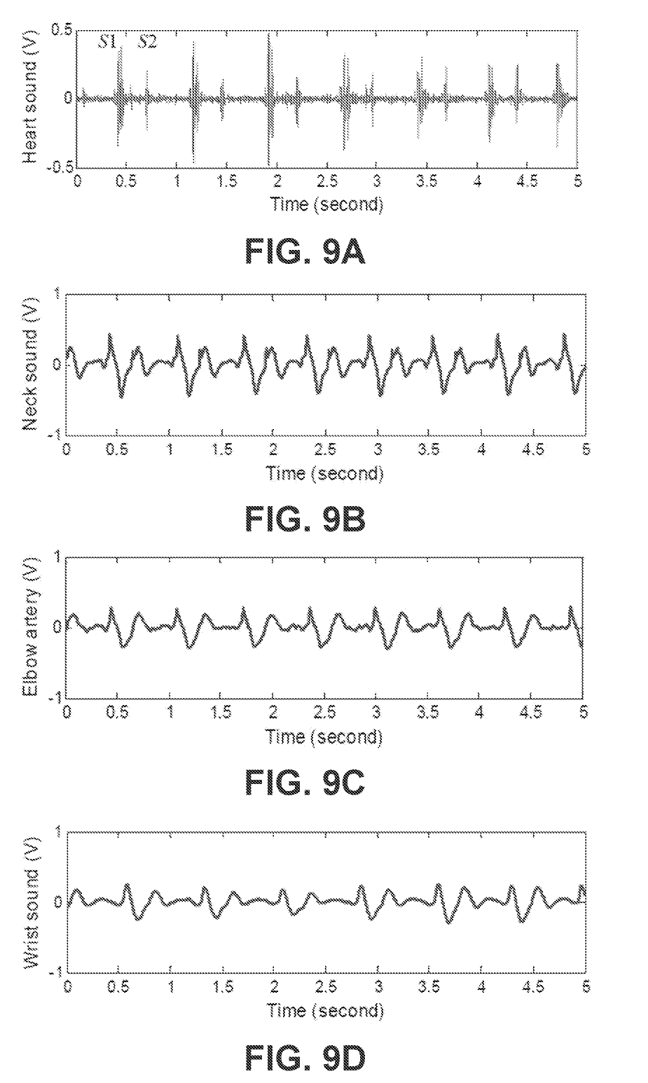

Pulse wave sounds were first simultaneously captured from the test subject's heart and the left subclavian artery by placing the stethoscope head on the chest and placing the air chamber on top of the left side of the neck. The signals acquired from the stethoscope head and air chamber are shown in FIGS. 9A and 9B, respectively. The air chamber was then moved to the left elbow to simultaneously acquire the arterial pulse wave sounds from the brachial artery near the left elbow and the heart sounds from the chest. The signals acquired from brachial artery are shown in FIG. 9C. Finally, the chamber was placed on the wrist to simultaneously acquire the arterial pulse wave sounds from the radial artery and the heart sounds from the chest. The signals acquired from the radial artery are shown in FIG. 9D.

Important acoustic properties of the heart sounds, such as the occurring time of S1 and S2, can be calculated from the time-frequency peaks of FIG. 9A. The acoustic properties, S1 and S2, transient heart rate, and the ratio of S1 and S2 are listed in Table 3, which can be used to continuously monitor for arrhythmia and other heart conditions.

TABLE 3
Acoustic Properties of the Heart Sound

| Heart sound | | | | | | | |
|---|---|---|---|---|---|---|---|
| segment (n) | 1 | 2 | 3 | 4 | 5 | 6 | Mean |
| Occurring time of | 0.42 | 1.17 | 1.92 | 2.68 | 3.42 | 4.13 | N/A |
| S1 (seconds) | | | | | | | |
| Occurring time of | 0.71 | 1.46 | 2.21 | 2.96 | 3.70 | 4.41 | N/A |
| S2 (seconds) | | | | | | | |
| S1(n + 1) − S1(n) | N/A | 0.75 | 0.75 | 0.76 | 0.74 | 0.71 | 0.74 |
| (seconds) | | | | | | | |
| Heart rate (bpm) | N/A | 80.00 | 80.00 | 78.95 | 81.08 | 84.51 | 80.91 |
| S2(n) − S1(n) | 0.29 | 0.29 | 0.29 | 0.28 | 0.28 | 0.28 | 0.28 |
| (seconds) | | | | | | | |
| S2 to S1 ratio | 38.67 | 38.67 | 38.16 | 37.84 | 39.44 | N/A | 38.56 |
| $\frac{S 2 ( n ) - S 1 ( n )}{S 1 ( n + 1 ) - S 1 ( n )}$ | % | % | % | % | % | | % |

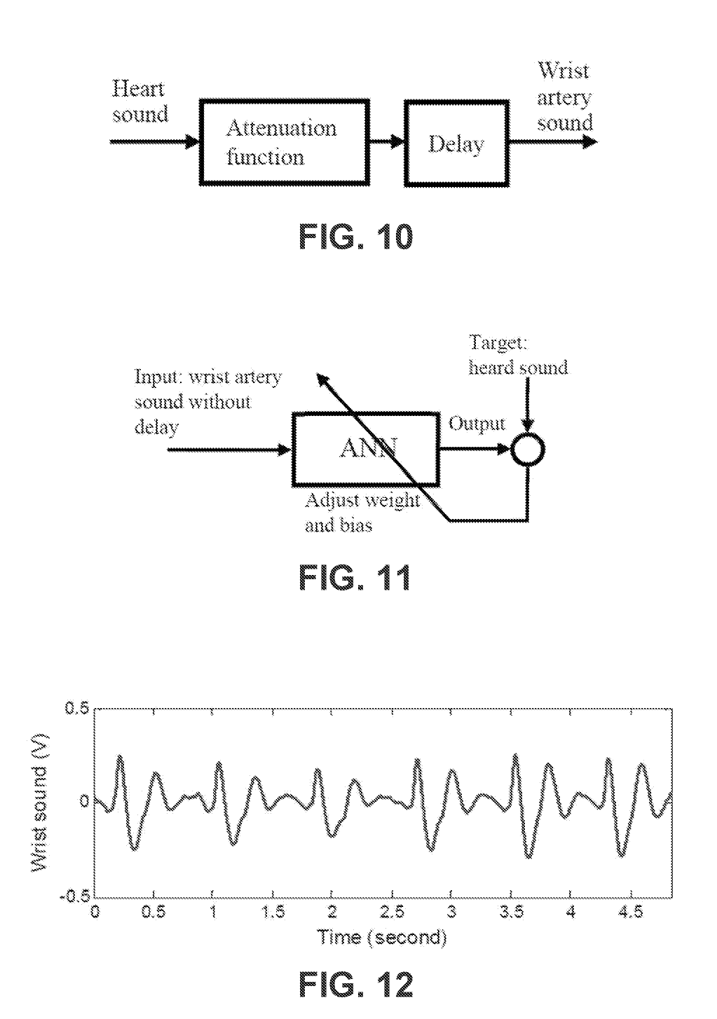

As is apparent from FIG. 9, the arterial pulse waveforms attenuate with the increased time delays from the heart to the left subclavian artery, connecting from the heart and the neck, to the brachial artery, connecting to the elbow, and radial artery at the wrist. The sounds travel in blood vessels and can be modeled with a time delay block and an attenuation function block, as shown in FIG. 10.

The time delay between the heart sounds and the various arterial pulse wave sounds can be estimated by pulse peaks in the time domain. In some embodiments, the sound delays of various arteries can be estimated by time-frequency peaks using STFT, which can give accurate transient occurrence times of S1 and S2. The estimated time delay from the heart to the subclavian artery at the neck is 0.05 seconds, from the heart to the brachial artery at the elbow is 0.095 seconds, and from the heart to radial artery at the wrist is 0.155 seconds. The distance from the test subject's heart to his neck was 0.25 m, the distance from the test subject's heart to his elbow was 0.48 m, and the distance from the test subject's heart to his wrist was 0.78 m. The average velocity of the test subject's pulse wave was estimated as about 5 m/s.

A two-layer, feed-forward, backpropagation artificial neural network (FIG. 11) was employed and trained. The network had a hidden layer with 500 tansig (hyperbolic tangent sigmoid transfer function) neurons and an output layer with one linear neuron. Using the Levenberg-Marquardt algorithm, the network was trained to adjust its weight and bias. Arterial pulse wave sound signals taken from the radial artery at the wrist shown in FIG. 12 were shifted to eliminate the estimated time delay and were then were serially fed into the neural network. The heart sounds shown in FIG. 9A were used as the target for training the neural network. The inverse attenuation function shown in FIG. 10 was emulated by the neural network. The training error was measured by mean squared error (MSE) expressed in Equation 3:

$$MSE = \sqrt{\frac{1}{N} {\sum }_{i = 1}^{N} {[ {x}_{O} ( N ) - {x}_{T} ( N ) ]}^{2}} \qquad ( 3 )$$

where x_{O} is the network output, x_{T} is the target, and N is sample number. After 20 training iterations, the mean squared error between the network output and the target was less than 0.1.

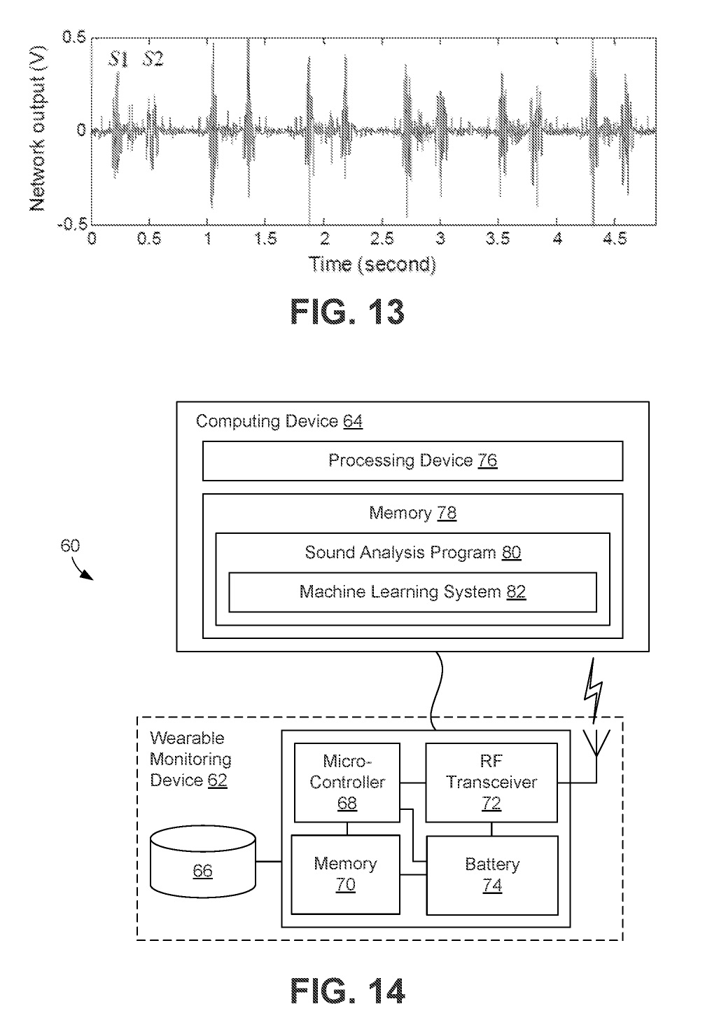

The trained network can be used to express the transfer function of the attenuation process for estimating heart sounds. An example of trained network outputs for the arterial pulse wave sounds obtained from the radial artery at the wrist without the delay as input are shown in FIG. 13. The distances of S1 and S2 of the estimated heart sounds as well as detailed acoustic properties can be calculated from the time-frequency peaks. The results of such calculations are listed in Table 4.

TABLE 4
Acoustic Properties of the Estimated Heart Sound

| Heart sound | | | | | | | | Mean |
|---|---|---|---|---|---|---|---|---|
| segment (n) | 1 | 2 | 3 | 4 | 5 | 6 | Mean | error |
| Occurring time of S1 | 0.43 | 1.19 | 1.93 | 2.69 | 3.42 | 4.14 | N/A | N/A |
| (seconds) | | | | | | | | |
| Occurring time of S2 | 0.70 | 1.46 | 2.21 | 2.98 | 3.70 | 4.43 | N/A | N/A |
| (seconds) | | | | | | | | |
| S1(n + 1) − S1(n) | N/A | 0.76 | 0.74 | 0.76 | 0.73 | 0.72 | 0.74 | 0.00 |
| (seconds) | | | | | | | | |
| Heart rate (bpm) | N/A | 78.95 | 81.08 | 78.95 | 82.19 | 83.33 | 80.90 | −0.01 |
| S2(n) − S1(n) | 0.27 | 0.27 | 0.28 | 0.29 | 0.28 | 0.29 | 0.28 | 0.00 |
| (seconds) | | | | | | | | |
| S2 to S1 ratio | 35.53 | 36.49 | 36.84 | 39.73 | 38.89 | N/A | 37.50 | −1.06 |
| $\frac{S 2 ( n ) - S 1 ( n )}{S 1 ( n + 1 ) - S 1 ( n )}$ | % | % | % | % | % | | % | % |

Comparison of the waveforms shown in FIGS. 9A and 13, as well as the acoustic properties of the heart sounds listed in Tables 3 and 4, reveals that the outputs of the trained network approximate the original heart sounds well. The mean errors in Table 4, as the differences of means in Tables 3 and 4, are small. It is, therefore, clear that accurate training results have been obtained.

In view of the foregoing discussion, it can be appreciated that an individual's heart sounds can be approximated from arterial pulse wave sounds captured from locations near an artery. The sounds can be captured from substantially any artery from which sound signals can be obtained. One convenient location is the radial artery at the wrist given that a wearable device can be easily integrated into a device, such as watch or wrist band, that can be comfortably worn on the wrist for extended periods of time for continuous monitoring. FIG. 14 illustrates an example system 60 for monitoring heart activity that comprises such a device.

With reference to FIG. 14, the system 60 generally comprises a wearable monitoring device 62 and a computing device 64. The monitoring device 62 can be worn in any location at which arterial pulse wave sounds can be captured. In some embodiments, the monitoring device 62 is configured for wearing on the wrist adjacent the radial artery. In other embodiments, the monitoring device 62 is configured for wearing around the neck adjacent the subclavian artery. In further embodiments, the monitoring device 62 is configured for wearing around the upper arm adjacent the brachial artery. In still further embodiments, the monitoring device 62 is configured for wearing around the upper thigh adjacent the femoral artery. The monitoring device 62 can comprise attachment means, such as a strap or adhesives, which are appropriate for attachment to the part of the body on which the device is to be worn. In cases in which the monitoring device 62 is worn on the wrist, the device can be incorporated into or integrated with another device commonly worn on the wrist, such as a watch, bracelet, or digital health monitor.

Irrespective of which part of the body on which the monitoring device 62 is to be worn, the device includes a sensor 66 that can be applied to the skin for the purpose of capturing arterial pulse wave sounds. In some embodiments, the sensor 66 comprises a microphone. In such cases, the microphone can be mounted within an air chamber that separates the pickup element of the microphone from the skin with an air chamber so as to reduce noise. In other embodiments, the sensor 66 can comprise a transducer, such as a piezoelectric or piezoresistive transducer, that can be applied directly to the skin.

The wearable monitoring device 62 further includes various other electrical components, which can include a microcontroller 68, memory 70, an RF transceiver 72, and a battery 74. The microcontroller 68 converts the analog signals captured by the sensor 66 into digital signal that can be stored in memory 70 as well as provided to the transceiver 72 for wireless transmission to the computing device 64. In some embodiments, the data collected by the sensor 66 can be stored locally in memory 70 and intermittently transmitted to the computing device 64. In other embodiments, the data collected by the sensor 66 can be transmitted to the computing device 64 in real time. While the monitoring device 62 is shown as including an RF transceiver 72, it is noted that, in some embodiments, the device can transmit data to the computing device 64 using a wired connection.

The computing device 64 can comprise any device that is capable of receiving, storing, and/or analyzing the signals from the wearable monitoring device 62. In some embodiments, the computing device 30 is a portable computing device, such as a laptop computer, tablet, or smart phone, so that it can be carried with the user to enable long-term data collection.

With further reference to FIG. 14, the computing device 64 comprises a processing device 76 and memory 78 (a non-transitory computer-readable medium). The memory 78 stores a sound analysis program 80, comprising one or more algorithms that are configured to analyze the signals from the wearable monitoring device 62. More particularly, the algorithms are configured to receive arterial pulse wave sound data from the monitoring device 62 and use a transfer function to estimate the parameters of the individual's heart sounds, such as S1 and S2. In some embodiments, the program 80 does this using a machine learning algorithm 82, such as an artificial neural network. Although an artificial neural network has been identified, it is noted that other machine learning systems can be used.

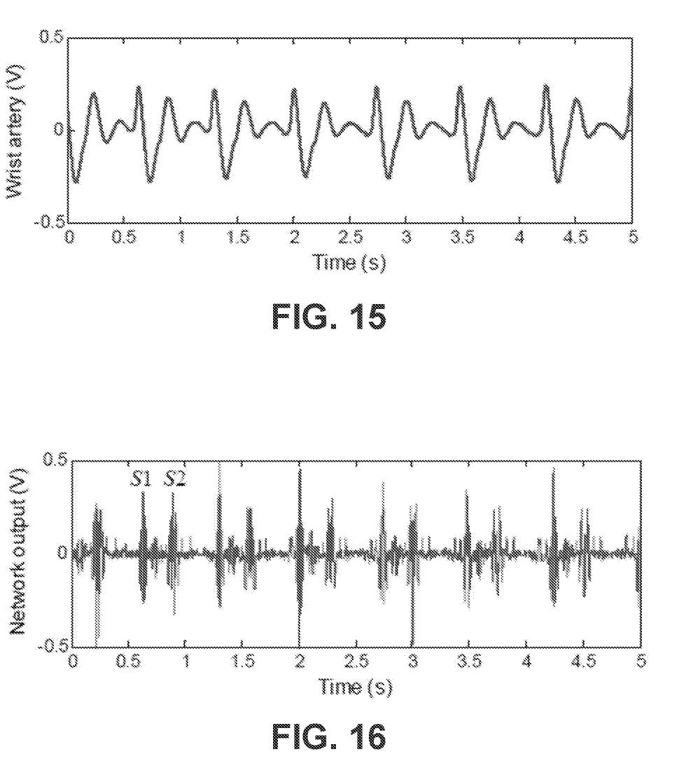

FIG. 15 shows signals that were obtained from the radial artery at the wrist using a monitoring device placed on left radial artery of the subject's wrist. FIG. 16 shows the heart sounds that were estimated from the signals of FIG. 15 using a trained network. The distances of S1 and S2 of the estimated heart sounds can been calculated by the time-frequency peaks again. The acoustic properties are listed in Table 5. The measurement and estimation results demonstrate the feasibility of a monitoring device worn on the wrist for heart sounds monitoring.

TABLE 5
Acoustic Properties Of The Verified Hear Sound Using A Bluetooth
Module

| Heart sound segment | | | | | | | |
|---|---|---|---|---|---|---|---|
| (n) | 1 | 2 | 3 | 4 | 5 | 6 | Mean |
| Occurring time of S1 | 0.57 | 1.30 | 2.01 | 2.74 | 3.48 | 4.24 | N/A |
| (seconds) | | | | | | | |
| Occurring time of S2 | 0.89 | 1.61 | 2.30 | 3.03 | 3.77 | 4.54 | N/A |
| (seconds) | | | | | | | |
| S1(n + 1) − S1(n) | N/A | 0.73 | 0.71 | 0.73 | 0.74 | 0.76 | 0.73 |
| (seconds) | | | | | | | |
| Heart rate (bpm) | N/A | 82.19 | 84.51 | 82.19 | 81.08 | 78.95 | 82.19 |
| S2(n) − S1(n) (seconds) | 0.32 | 0.31 | 0.29 | 0.29 | 0.29 | 0.30 | 0.30 |
| S2 to S1 ratio | 43.84 | 43.66 | 39.73 | 39.19 | 38.16 | N/A | 40.91 |
| $\frac{S 2 ( n ) - S 1 ( n )}{S 1 ( n + 1 ) - S 1 ( n )}$ | % | % | % | % | % | | % |

1. A system for monitoring heart activity comprising:
a wearable monitoring device including a sensor adapted to capture arterial pulse wave sounds; and
a computing device configured to receive arterial pulse wave sound data from the wearable monitoring device and estimate heart sounds from the data.

2. The system of claim 1, wherein the wearable monitoring device is configured to be worn on the wrist adjacent the radial artery.

3. The system of claim 2, wherein the wearable monitoring device further includes attachment means for attaching the device to the wrist.

4. The system of claim 1, wherein the sensor comprises a microphone.

5. The system of claim 4, wherein the sensor further comprises an air chamber associated with the microphone.

6. The system of claim 1, wherein the sensor comprises a transducer.

7. The system of claim 1, wherein the wearable monitoring device further includes a microcontroller that digitizes the arterial pulse wave sounds to generate the arterial pulse wave sound data.

8. The system of claim 7, wherein the wearable monitoring device further includes a transceiver that is configured to wirelessly transmit the arterial pulse wave sound data to the computing device.

9. The system of claim 1, wherein the computing device is configured to estimate S1 and S2 sounds from the arterial pulse wave sound data.

10. The system of claim 1, wherein the computer is configured to estimate the heart sounds from the arterial pulse wave sound data using a transfer function.

11. The system of claim 10, wherein the transfer function is emulated by a machine learning system.

12. The system of claim 11, wherein the machine learning system comprises a trained artificial neural network.

13. A method for monitoring heart sounds, the method comprising:
a user wearing a wearable monitoring device on a location of the body adjacent an artery;
capturing arterial pulse wave sounds with the wearable monitoring device;
transmitting digitized arterial pulse wave sound data to a computing device; and
estimating heart sounds from the arterial pulse wave sound data using the computing device.

14. The method of claim 13, wherein wearing a wearable monitoring device comprises wearing the monitoring device on the wrist.

15. The method of claim 14, wherein capturing arterial pulse wave sounds comprises capturing the arterial pulse wave sounds from the radial artery.

16. The method of claim 13, wherein transmitting digitized arterial pulse wave sound data comprises wirelessly transmitting the data from the wearable monitoring device to the computing device.

17. The method of claim 13, wherein estimating heart sounds comprises estimating S1 and S2 sounds from the arterial pulse wave sound data.

18. The method of claim 13, wherein estimating heart sounds comprises estimating the heart sounds from the arterial pulse wave sound data using a transfer function.

19. The method of claim 13, wherein estimating heart sounds comprises inputting the arterial pulse wave sound data into a machine learning system trained to convert the arterial pulse wave sound data into heart sounds.

20. The method of claim 13, wherein estimating heart sounds comprises inputting the arterial pulse wave sound data into an artificial neural network trained to convert the arterial pulse wave sound data into heart sounds.
